(19) United States
(12) Patent Application Publication
(10) Pub. No.: US 2023/0068943 A1
(43) Pub. Date: Mar. 2, 2023

(54) FLOW BLOCKING CATHETER

(71) Applicant: MICROPORT NEUROTECH (SHANGHAI) CO., LTD., Shanghai (CN)

(72) Inventors: Yuxi CUN, Shanghai (CN); Yunyun LIU, Shanghai (CN); Guanyu LIN, Shanghai (CN); Yumei LIU, Shanghai (CN)

(21) Appl. No.: 17/460,821

(22) Filed: Aug. 30, 2021

(51) Int. Cl. A61M 25/10; A61M 25/00; A61L 29/08

(52) U.S. Cl. CPC A61M 25/104; A61M 25/10185; A61M 25/0012; A61M 25/0045; A61L 29/085

(57) ABSTRACT

A flow blocking catheter including an inner tube, a flow blocking member and an outer tube is provided. The flow blocking member is self-expandable and sleeved on an exterior of the inner tube. At least a proximal end of the self-expandable flow blocking member is attached to an outer circumference of the inner tube. The outer tube is movably sleeved on the exterior of the inner tube to restrict an expansion of the flow blocking member. In this way, expansion of the flow blocking member is able to be controlled simply by pushing/retracting the outer or inner tube to offer a fast shifting between different configurations. The flow blocking member is able to occlude blood flow with a controllably expansion to lower stimulation to the wall of the blood vessel and avoid the easy bursting of the balloon.

### TECHNICAL FIELD

The present application relates to the field of medical instruments and, in particular, to a flow blocking catheter.

### BACKGROUND

Strokes, mainly caused by blood clots in cerebral blood vessels, are a common medical condition that seriously threatens human health, which is also the third leading cause of death worldwide and the leading cause of long-term disability in adults. In the current clinical practice, treatments of directly sucking the thrombus with an aspiration catheter or removing the thrombus with the assistance of a stent are used to eliminate the thrombus to achieve recanalization of the blood vessel. After the aspiration catheter reaches the thrombus sit along the blood vessel, a negative pressure is applied at its proximal end to suck the clot into the aspiration catheter or onto the opening of aspiration catheter, followed by slow retraction of the clot into a guide catheter. As a result, the blood vessel recovers back to its normal hemodynamic condition. The thrombectomy stent is required to cross over the clot, trap the clot within meshes of the stent and then retract back into the support catheter, so as to recanalize the blood vessel. After the stent is retracted back into the support catheter, the support catheter together with the stent and blood clot trapped in the stent, is in turn withdrawn into the guide catheter. However, during the thrombus removal process, the fragment clots often fall off and are rushed to the distal blood vessel due to the impact of proximal blood flow, or during the operation of the aspiration catheter or the delivery of thrombectomy stent into the interventional instrument (the guide or support catheter) after the succesful capture of clots, the clots break up to creat clot fragments that are rushed to the distal blood vessel by the blood flow to cause secondary blocking, which results in the failure of operation and may even threaten the patient's life in severe cases. For example, the possibility of percutaneous coronary intervention (PCI) caused myocardial necrosis reaches as high as 16%-39%, and most of these cases have been found to be attributable to escape of clots into distal blood vessels during the intervention operation. In order to solve the problems caused by clot fragmentation, the balloon guide catheter has been adpoted commonly in prior art to facilitate the thrombus removal operation by temporarily occluding the blood flow.

Typically, during the operation, after a thrombus removal device is delivered to a target site with the assistance of a balloon guide catheter (i.e., an aspiration or support catheter passes through a lumen of the balloon guide catheter to reach the target site), the balloon is expanded against the blood vessel wall by injecting a contrast fluid in the balloon so as to temporarily occlude blood flow in the vessel. Moreover, after the blood clot has been taken into a lumen of the aspiration or support catheter, the balloon is contracted, followed by withdrawal of the balloon guide catheter. In this way, the blood clot is taken out of the patient's body to achieve the effect of blood flow reconstruction.

However, in existing balloon guide catheters, the balloon is typically provided on the outside of an outer tube. As the balloon has a certain thickness itself and given that an outer diameter of the catheter must be designed to be not too large to ensure its smooth passage in blood vessels, the catheter has to assume a very small inner diameter, making it impossible to be fitted with a wide-lumen aspiration or support catheter. This therefore makes it unable to treat large-size thrombi. Moreover, for balloon guide catheter, since it is necessary to fill the balloon with radiopaque or other liquid to make the balloon bulge and attach to the blood vessel wall to block the blood flow, it may take some time to achieve a complete blood flow blocking effect. It may also be the case for the withdrawal of the balloon guide catheter by drawing out the radiopaque fluid. This not only prolongs the surgical time, but may also lead to ischemia or even necrosis of the tissue due to an excessively long blood flow blocking time. This may also resuls in a risk of blood vessel damage from over-expansion or bursting of the balloon. More importantly, during surgery, if it is found that the balloon is dilated at an improper location, the radiopaque fluid has to be completely discharged before the balloon can be relocated and re-expanded. This not only takes much more time but also increases risk of bursting of the balloon to cause secondary damage to the blood vessel due to the repeated dilation. Further, the pressure exerted by the dilated balloon tends to stimulate the wall of the cerebral blood vessel and thus cause various complications during the surgical procedure. All these shortcomings limit the benefits of using balloons in thrombus removal procedures, increase complexity of such procedures and expose the patients to high risk.

### SUMMARY

It is an object of the present application to provide a flow blocking catheter to overcome the problems of slow flow blocking, low safety and reliability, poor reproducibility and small catheter lumen of existing guide catheters that are brought by the use of balloon for blood flow blocking.

To solve the above problem, present application provides a flow blocking catheter comprising:

an inner tube;

a self-expandable flow blocking member sleeved on an exterior of the inner tube, at least a proximal end of the self-expandable flow blocking member attached to an outer circumference of the inner tube; and

an outer tube movably sleeved on the exterior of the inner tube to restrict expansion of the flow blocking member.

Optionally, in the flow blocking catheter, the flow blocking member comprises a support frame, and at least an proximal end of the support frame is attached to the outer circumference of the inner tube and the support frame is self-expandable.

Optionally, in the flow blocking catheter, the flow blocking member further comprises a flow blocking membrane attached to the support frame.

Optionally, in the flow blocking catheter, the flow blocking member has its one end attached to the outer circumference of the inner tube and the other end to be a free end, or the flow blocking member has opposing ends attached to the outer circumference of the inner tube.

Optionally, in the flow blocking catheter, the outer circumference of the inner tube is provided with a groove that matches with a shape of the flow blocking member in a collapsed configuration to accommodate the flow blocking member.

Optionally, the flow blocking catheter further comprise a control valve configured to drive the outer or inner tube to move relative to the inner or outer tube.

Optionally, in the flow blocking catheter, the control valve comprises a control valve body and a control slider coupled to the control valve body, the control slider configured to be axially slidable. The control valve body is coupled to a proximal end of the inner tube with the control slider being coupled to a proximal end of the outer tube; or the control valve body is coupled to the proximal end of the outer tube with the control slider being coupled to a proximal end of the inner tube.

Optionally, in the flow blocking catheter, both or either of the inner tube and the outer tube is a single-layered tube made of macromolecular material.

Optionally, in the flow blocking catheter, both or either of the inner tube and the outer tube has a structure comprising at least two layers, in which both or either of a first layer and a second layer from inside to outside is made of macromolecular material.

Optionally, in the flow blocking catheter, both or either of the inner tube and the outer tube has a structure comprising at least two layers, in which a second layer from inside to outside comprises one or more selected from the group consisting of braided structure, coil, and cut hypotube.

Optionally, in the flow blocking catheter, each of the inner and outer tubes has a triple-layered structure.

Optionally, in the flow blocking catheter, the inner tube comprises a first radiopaque ring disposed at a distal end of the inner tube.

Optionally, in the flow blocking catheter, the inner tube further comprises a second radiopaque ring disposed at a location of the inner tube where the flow blocking member is attached to the inner tube.

Optionally, in the flow blocking catheter, the flow blocking member has one end attached to the outer circumference of the inner tube and the other end to be an free end, and the flow blocking member further comprises a third radiopaque ring disposed at the free end thereof.

Optionally, in the flow blocking catheter, the flow blocking member comprises at least one selected from the group consisting of mesh structure, open-loop structure and spiral structure, and the flow blocking member is fabricated by braiding, winding or cutting.

Optionally, in the flow blocking catheter, the mesh structure is braided from 1-64 filaments. The filament is at least one selected from the group consisting of regular filament, radiopaque filament and composite filament. The regular filaments is made of at least one selected from the group consisting of nickel-titanium alloy, cobalt-chromium alloy, stainless steel and macromolecular material. The radiopaque filaments is made of at least one selected from the group consisting of radiopaque metal, alloy of radiopaque metals and macromolecular material containing a radiopaque agent. The composite filament is formed by a radiopaque core filament combined with a regular filament.

Optionally, the flow blocking catheter further comprises a securing film that is secured and attached to an exterior of the proximal end of the flow blocking member and at least covers a part of the flow blocking member and a part of the inner tube.

In summary, the flow blocking catheter of the present application comprises an inner tube, a flow blocking member and an outer tube, the flow blocking member being self-expandable and sleeved on an exterior of the inner tube, at least a proximal end of the self-expandable flow blocking member attached to an outer circumference of the inner tube, the outer tube movably sleeved on the exterior of the inner tube to restrict an expansion of the flow blocking member. With this configuration, expansion of the flow blocking member is able to be controlled simply by pushing/retracting the outer or inner tube, which allows to achieve a fast shifting between different configurations, relocatability during a surgical procedure, and simple and time-saving operation. In addition, the flow blocking member is able to occlude blood flow with a controllably expansion, thereby lowering stimulation to the wall of the blood vessel while avoiding the problem of easy bursting arising from the use of a balloon. Moreover, the flow blocking member has a small thickness when in a collapsed configuration, allowing an increased inner diameter of the catheter at a given outer diameter of the flow blocking catheter and thus making it applicable to the treatment of large blood clots or passage of large instruments.

### BRIEF DESCRIPTION OF THE DRAWINGS

Those of ordinary skill in the art would appreciate that the appended figures are presented merely to enable a better understanding of the present application rather than limit the scope thereof in any sense. In the figures,

FIG. 1 is a schematic diagram of a flow blocking catheter according to a preferred embodiment of the present application;

FIG. 2 is a schematic cross-sectional view of an inner tube according to a preferred embodiment of the present application;

FIG. 3 is a schematic diagram of an expanded flow blocking member according to a preferred embodiment of the present application;

FIG. 4 is a schematic diagram of a control valve according to a preferred embodiment of the present application;

FIG. 5 is a schematic cross-sectional view of a flow blocking catheter according to a preferred embodiment of the present application;

FIG. 6 is a schematic diagram of a flow blocking catheter provided with an groove according to a preferred embodiment of the present application;

FIG. 7 is a schematic diagram of a flow blocking catheter provided with a securing film according to a preferred embodiment of the present application;

FIG. 8 is a schematic diagram of a flow blocking member having its two ends attached to an inner tube respectively according to a preferred embodiment of the present application;

FIG. 9 is a schematic diagram of a braided structure of a flow blocking member according to a preferred embodiment of the present application; and

FIGS. 10a to 10g schematically illustrate various mesh openings of support frames according to preferred embodiments of the present application.

In the Figures,
100, inner tube; 101, first layer; 102, second layer; 103, third layer; 104, adhesive; 110, groove; 120, first radiopaque ring;
200, outer tube; 210, distal end of the outer tube; 220, stress dispersion tube;
300, flow blocking member; 310, first end; 320, second end; 330, radiopaque filament; 340, mesh opening;
400, control valve; 410, control valve body; 420, control slider; 430, sliding slot; 440, catheter insertion opening; 500, securing film.

### DETAILED DESCRIPTION

To make objects, advantages and features of the present application more apparent, present application is described in detail by the particular embodiments made in conjunction with the accompanying drawings. Note that the figures are provided in a very simplified form not necessarily drawn to exact scale, with the only intention to facilitate convenience and clarity in explaining the present application. In addition, structures shown in the figures are usually part of actual structures. In particular, as the figures tend to have distinct emphases, they are often drawn to different scales.

As used in present specification, the meaning of “a,” “an,” and “the” include singular and plural references, unless the context clearly dictates otherwise. As used in present specification and appended claims, the term “or” genreally includes the meaning of “and/or”, unless the context clearly dictates otherwise. Additionally, the terms “proximal” and “distal” are generally used to refer to an end close to an operator and an end close to a lesion site in a patient, respectively. Further, the terms “one end” and “the other end”, or “proximal end” and “distal end”, are generally used to refer to two opposing portions including not only the endpoints.

The core idea of the present application is to provide a flow blocking catheter to overcome the problems of slow flow blocking, low safety and reliability, poor reproducibility and small catheter lumen of existing guide catheters that are brought by the use of balloon for blood flow blocking. The flow blocking catheter comprises an inner tube, a flow blocking member and an outer tube, the flow blocking member being self-expandable and sleeved on an exterior of the inner tube, at least a proximal end of the self-expandable flow blocking member attached to an outer circumference of the inner tube, the outer tube movably sleeved on the exterior of the inner tube to restrict an expansion of the flow blocking member. With this configuration, expansion of the flow blocking member is able to be controlled simply by pushing/retracting the outer or inner tube, which allows to achieve a fast shifting between different configurations, few influence on tissue blood supply, relocatability during a surgical procedure, and simple and time-saving operation. In addition, the flow blocking member is able to occlude blood flow with a controllably expansion, thereby lowering stimulation to the wall of the blood vessel while avoiding the problem of easy bursting arising from the use of a balloon. Moreover, the flow blocking member has a small thickness when in a collapsed configuration, allowing an increased inner diameter of the catheter at a given outer diameter of the flow blocking catheter and thus making it applicable to the treatment of large blood clots or passage of large instruments.

In the following description, reference is made to FIGS. 1 to 10g.

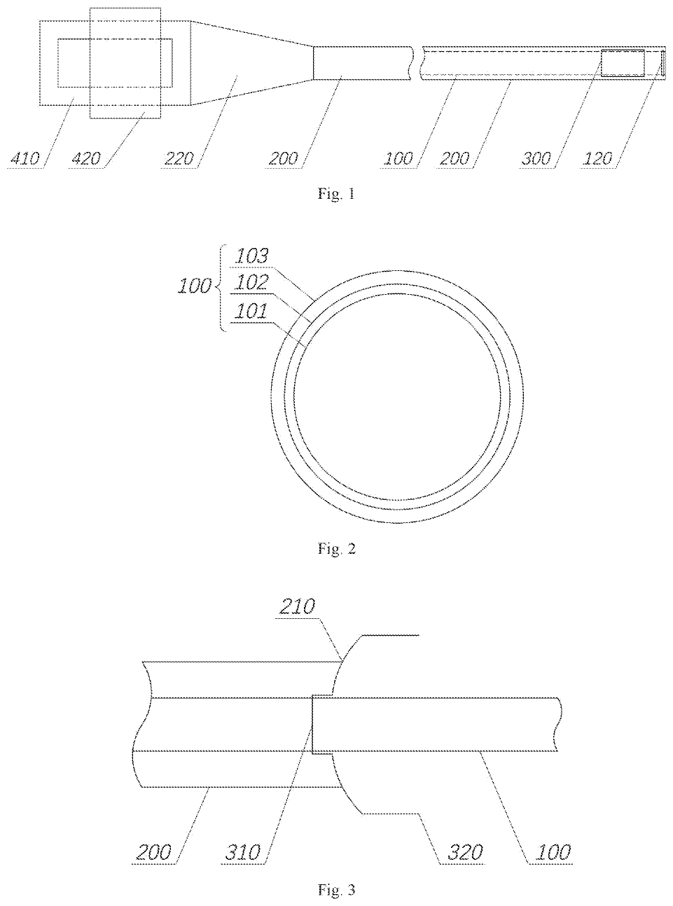

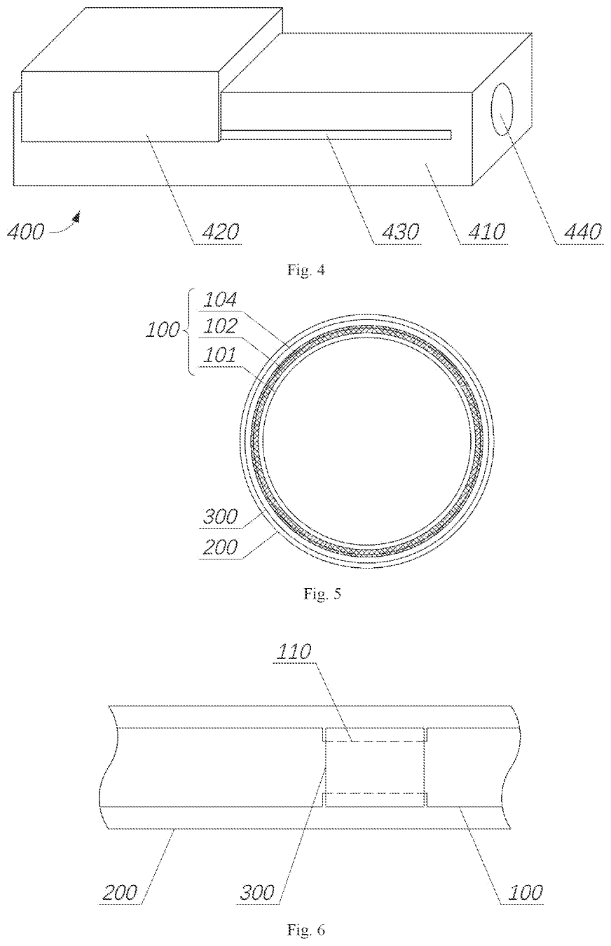

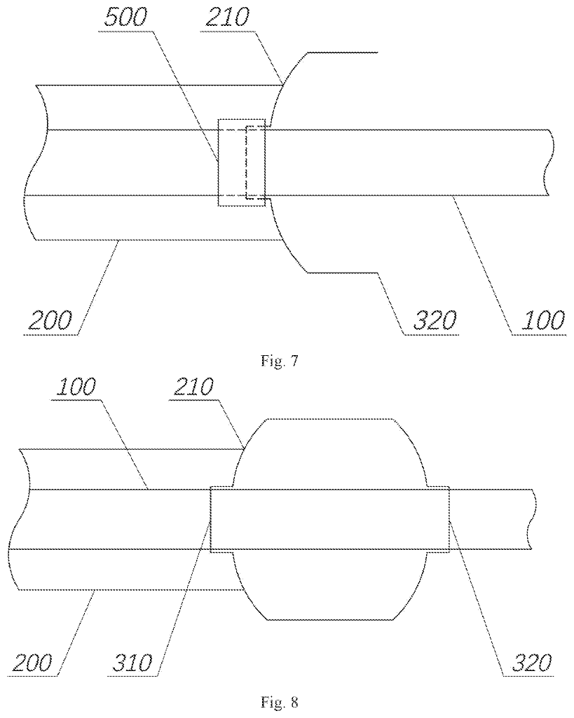

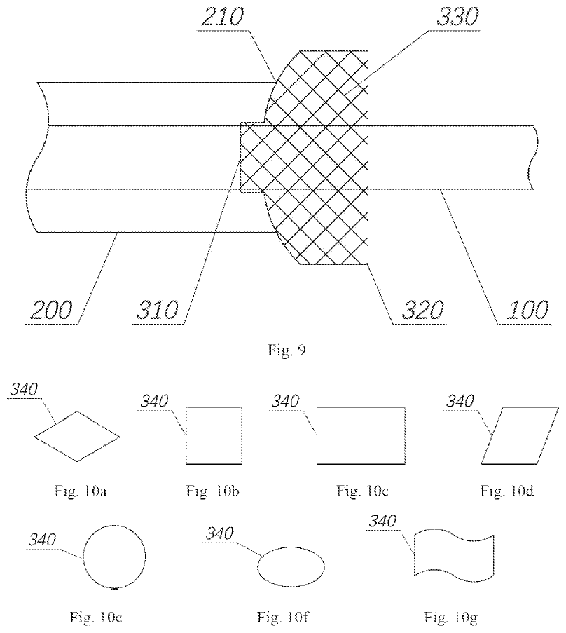

FIG. 1 is a schematic diagram of a flow blocking catheter according to a preferred embodiment of the present application. FIG. 2 is a schematic cross-sectional view of an inner tube according to a preferred embodiment of the present application. FIG. 3 is a schematic diagram of an expanded flow blocking member according to a preferred embodiment of the present application. FIG. 4 is a schematic diagram of a control valve according to a preferred embodiment of the present application. FIG. 5 is a schematic cross-sectional view of a flow blocking catheter according to a preferred embodiment of the present application. FIG. 6 is a schematic diagram of a flow blocking catheter provided with a groove according to a preferred embodiment of the present application. FIG. 7 is a schematic diagram of a flow blocking catheter provided with a securing film according to a preferred embodiment of the present application. FIG. 8 is a schematic diagram of a flow blocking member having its two ends attached to an inner tube respectively according to a preferred embodiment of the present application. FIG. 9 is a schematic diagram of a braided structure of a flow blocking member according to a preferred embodiment of the present application. FIGS. 10a to 10g schematically illustrate various mesh openings of support frames according to preferred embodiments of the present application.

As shown in FIGS. 1 and 2, a flow blocking catheter according to an embodiment includes an inner tube 100, a flow blocking member 300 and an outer tube 200. The flow blocking member 300 is self-expandable. The flow blocking member 300 is sleeved on the exterior of the inner tube 100. At least proximal end (the first end 310) of the flow blocking member 300 is attached (e.g., by adhesive bonding, welding or a securing film) to an outer circumference of the inner tube 100. The outer tube 200 is movably sleeved on the exterior of the inner tube 100 in order to restrict expansion of the flow blocking member 300. In some embodiments, the flow blocking member 300 is configured as that the outer tube 200 moves toward a proximal end of the inner tube 100 to remove its restriction to the flow blocking member 300, so that the flow blocking member 300 expands (radially) due to its own expandability; the outer tube 200 moves toward a distal end of the inner tube 100 to restrict expansion of the flow blocking member 300, so that the flow blocking member 300 contracts (and recovers along a radial direction).

In another embodiments, the expansion and contraction of the flow blocking member 300 may be controlled by movements of the inner tube 100 relative to the outer tube 200. In this embodiment, the flow blocking member 300 has a first end 310 arranged close to the distal end of the inner tube 100 so that the flow blocking occurs at a location close to where a thrombus removal or other instrument operates, thus reducing adverse impact on blood flow around the proximal end. In alternative embodiments, the first end 310 of the flow blocking member 300 may be arranged at the middle or proximal end of the inner tube 100.

In one exemplary embodiment, both the inner 100 and outer 200 tubes are preferred to be circular tubes and the outer tube 200 is sleeved on the inner tube 100. The difference between an outer diameter of the inner tube 100 and an inner diameter of the outer tube 200 may range from 0.0001 inch to 0.1 inch. The outer tube 200 is preferred to be a single-layered tubular member formed of, for example, one or more of a polyether-polyamide block copolymer (PEBA or Pebax), polyamide (PA) and polytetrafluoroethylene (PTFE). The inner tube 100 includes at least a single macromolecular layer made of a macromolecular material that may be one or more selected from the group consisting of PTFE, high-density polyethylene (HDPE), Pebax mixed with a friction coefficient reducing additive, and polyolefin elastomer (POE). Preferably, the inner tube 100 includes a triple-layered structure, as shown in FIG. 5, consisting of a first layer 101, a second layer 102, and a third layer 103 arranged from inside to outside. The third layer 103 may be formed of, for example, one or more of the nylon elastomer (e.g., Pebax), nylon and polyurethane (PU). The first layer 101 may be formed of, for example, one or more of PTFE, HDPE, Pebax mixed with a friction coefficient reducing additive, and POE. The second layer 102 may consist of any one of a braided structure, a coil and a cut hypotube (here, the term “hypotube” refers to any metal tube for medical use), or a combination of two or more thereof. The second layer 102 may be formed of stainless steel, nickel-titanium alloy, cobalt-chromium alloy or macromolecular wire, which can increase force transmission performance, ellipticity resistance and bending resistance of the inner tube 100 as well as reduce a force required to withdraw the flow blocking member 300. It is to be understood that materials of the layers of the inner tube 100 are not limited to the materials listed above, and those skilled in the art may also choose other functionally similar materials based on prior art. As shown in FIG. 5, in one alternative embodiment, the inner tube 100 includes only two layers, which are a first layer 101 and a second layer 102 covered on the first layer. In this case, the first layer 101 may be essentially a macromolecular layer formed of one or more of PTFE, HDPE, Pebax mixed with a friction coefficient reducing additive, and POE. The second layer 102 may be essentially a metallic layer consisting of, for example, any one of a braided structure, a coil and a cut hypotube, or a combination of two or more thereof. The second layer 102 may be formed of, for example, stainless steel, a nickel-titanium alloy, a cobalt-chromium alloy or the like. Preferably, a layer of adhesive 104 may be applied onto the macromolecular layer, which penetrates into the metallic layer (i.e., part of the adhesive 104 penetrates through meshes formed in the metallic layer and adheres to the exterior of the macromolecular layer) to form a firm adhesion between the metallic and macromolecular layers, so as to improve the force transmission performance and ellipticity resistance. Of course, the outer tube 200 is not limited to being a single-layered tube in other embodiments, and it may also be implemented as a double-, triple- or more-layered structure. The specific structure of the outer tube 200 can refer to that of the inner tube 100.

Preferably, the inner tube 100 includes a first radiopaque ring 120 disposed at the distal end of the inner tube 100. In particular, the first radiopaque ring 120 may be disposed at a distal end of the second layer 102 in the inner tube 100. More preferably, the inner tube 100 further includes a second radiopaque ring (not shown) disposed at a location of the inner tube 100 where the flow blocking member 300 is attached to the inner tube 100. Further, when one end of the flow blocking member 300 is attached to the outer circumference of the inner tube 100 and the other end of the flow blocking member 300 is a free end, the flow blocking member 300 further includes a third radiopaque ring (not shown) disposed at the free end thereof. The design of the third radiopaque ring allows to visually reflect to what extent the free end of the flow blocking member 300 expands. Optionally, examples of materials of the first, second and third radiopaque rings may include, but are not limited to, platinum, iridium, tantalum, noble metal alloys and macromolecular materials containing radiopaque agents. Arranging the three radiopaque rings helps the operator locate the inner tube 100 during a surgical procedure, or enables visual reflection of expansion extent of the flow blocking member 300. It is to be understood that the first radiopaque ring 120 is located at the distal end of the inner tube 100, but it is not intended to limit that the first radiopaque ring 120 can only be located at the distal end face of the inner tube 100, which can be located in an area close to the distal end of the inner tube 100. While the above embodiment exemplifies the positions of the three radiopaque rings, it is not intended to limit that the three radiopaque rings must be porvided simultaneously, and those skilled in the art may select to provide any one or two of them according to the actual circumstances.

Preferably, the flow blocking member 300 includes a support frame, which is attached at least at its proximal end to the outer circumference of the inner tube 100 and is self-expandable. Optionally, the flow blocking member 300 may further include an flow blocking membrane attached to the support frame. In one example, the support frame is a tubular member that is able to switch between a collapsed configuration and an expanded configuration under the restriction of the outer tube 200. It is to be understood that the support frame is not limited to switch only between the collapsed configuration and the expanded configuration. In some cases, it may also assume an intermediate configuration between the collapsed and expanded configurations (i.e., a semi-expanded or partially-expanded configuration). The support frame may be formed of, for example, nickel-titanium alloy, Type 304 stainless steel, platinum-tungsten alloy, platinum-iridium alloy, cobalt-chromium alloy, radiopaque metal or the like. The support frame may be fabricated by winding, cutting or braiding. In this embodiment, the support frame includes a plurality of mesh openings 340, as shown in FIGS. 10a to 10g. The mesh opening 340 may have a rhombic (10a), square (10b), rectangular (10c), parallelogramic (10d), polygonal (not shown), circular (10e), elliptic (10f) or irregular (10g) shape, with the rhombic shape (10a) being preferred. The flow blocking membrane may be attached to either an inner surface or an outer surface of the support frame. The flow blocking membrane is preferably a macromolecular membrane formed of, for example, PU, polyethylene(PE), expanded polytetrafluoroethylene (ePTFE) or the like. It is to be understood that the support frame and the flow blocking membrane are not limited to being formed of the materials listed above, and those skilled in the art may also choose other functionally similar materials based on prior art. As shown in FIG. 9, in some embodiments, the support frame may be a mesh structure braided from 1-64 filaments. The filament may be at least one selected from the group consisting of regular filament, radiopaque filament and composite filament. The material of the regular filament may be selected as, but is not limited to, one or more of nickel-titanium alloy, cobalt-nickel alloy, stainless steel, macromolecular material and the like. The material of the radiopaque filament may be selected as, but is not limited to, radiopaque metal such as platinum, iridium, gold or tungsten, or the alloy thereof, or a macromolecular material containing a radiopaque agent of the radiopaque metal or alloy. The composite filament may be a bilayer structure consisting of a radiopaque core filament and a regular filament coated on the radiopaque core filament. The inner radiopaque core filament may be formed of one or more of a radiopaque metal such as platinum, iridium, gold or tungsten, or an alloy thereof, while the outer regular filament may be formed of one or more of the nickel-titanium alloy, cobalt-nickel alloy, stainless steel and macromolecular material. The radiopaque filament 330 imparts an improved radiographic visibility to the flow blocking member 300, and thus imparts an improved traceability to the flow blocking member 300 during use. In alternative embodiments, the support frame may be an open-loop structure or a spiral structure, or consist of two or more of a mesh structure, an open-loop structure and a spiral structure.

Referring to FIGS. 1 and 3, expansion state of the flow blocking member 300 (it would be appreciated that expansion state of the flow blocking member 300 is the same as that of the support frame) can be controlled by movement of the outer tube 200 along an axis of the inner tube 100. Specifically, in an initial configuration of the flow blocking catheter, the distal end 210 of the outer tube compresses the flow blocking member 300 to restrict expansion of the flow blocking member 300, where the flow blocking member 300 is in a collapsed configuration. Moreover, in this configuration, the flow blocking member 300 is compressed between the inner 100 and outer 200 tubes, facilitating movement of the flow blocking catheter in a blood vessel. When the outer tube 200 moves towards the proximal end relative to the inner tube 100 (i.e., the outer tube 200 is retracted), the flow blocking member 300 is exposed from the outer tube 200, and self-expands to press against the wall of a blood vessel. The blood flow in the blood vessel is thus blocked as a layer of the flow blocking membrane is attached to the support frame of the flow blocking member 300. It is to be understood that, at this point of time, the expansion of the flow blocking member 300 adapts to the dimension of the blood vessel wall, and the flow blocking member 300 is not necessarily fully expanded (i.e., flow blocking member 300 may be in a semi-expanded configuration). Of course, in some other cases, when the outer tube 200 retracts, the distal end 210 of the outer tube 200 may not separate from the flow blocking member 300 and only a part of the flow blocking member 300 is no longer restricted by the outer tube 200. Such part of the flow blocking member 300 thus self-expands to press against the blood vessel wall to block the blood flow. That is, the outer tube 200 may release and allow expansion of only part of the flow blocking member 300, but does not necessarily release and allow expansion of the entire flow blocking member 300 (i.e., the flow blocking member 300 may be in a partially-expanded configuration). Preferably, the flow blocking member 300 is compliant to a certain extent, which makes it able to adapt shapes of the blood vessel walls in an expanded configuration (including the fully-, semi- or partially-expanded configuration). This arrangement is favorable to blood vessels with vulnerable walls by reducing the force applied to these blood vessel walls from the expansion of the flow blocking member 300. As a result, the flow blocking member 300 is able to lower stimulation to the wall of a cerebral blood vessel, suppress the occurrence of various complications such asvasospasm during surgery and completely avoid the risk of secondary damage to the blood vessel caused by the bursting of a balloon or a balloon bonding section.

Further, when blood flow blocking has been attained, a blood clot can be directly sucked, or captured and pulled back via the lumen of the inner tube 100 (or a aspiration catheter may be deployed in the lumen of the inner tube 100 of the flow blocking catheter to suck the clot, or a support catheter may be deployed in the lumen, in which a thrombectomy stent is provided for removing the clot). As shown in FIG. 2, because of a relative small thickness of the flow blocking member 300 in the collapsed configuration, the lumen of the inner tube 100 takes up a much larger proportion of a cross-sectional area of the flow blocking catheter, when compared to the case of using a conventional balloon catheter. Therefore, for a given outer diameter, the flow blocking catheter of present application is able to be fitted with aspiration catheters or support catheters with large lumens, larger size stents or other medical instruments for treating large blood clots, while the outer diameter of the entire flow blocking catheter is limited to ensure that the flow blocking catheter is able to pass through tortuous distal blood vessels succesfully and causes a small wound to the patient.

Further, when it is necessary to change positions of the blood flow blocking by relocating or withdrawing the flow blocking catheter, the outer tube 200 may be caused to move proximally relative to the inner tube 100 (i.e., retracting the outer tube 200) until the distal end of the outer tube 200 comes into abutment against the flow blocking member 300, as shown in FIG. 3. Then, the outer tube 200 may be continuously pushed towards the distal end until the flow blocking member 300 is compressed into the collapsed configuration. The configuration of the flow blocking member 300 shown in FIG. 3 is reversible. That is, the flow blocking member 300 self-expands again, when the outer tube 200 is retracted proximally. The ability of the flow blocking member 300 to be collapsed repeatedly enables easy re-delivery and relocation of the flow blocking catheter. Therefore, the flow blocking catheter of this embodiment provides convenience for achieving repetitive operations, accurate location as well as convenience for achieving withdrawal from the blood vessel with the removed thrombus.

As shown in FIG. 4, the flow blocking catheter further includes a control valve 400 configured to drive movements of the outer tube 200 relative to the inner tube 100. In one embodiment, the control valve 400 includes a control valve body 410 and a control slider 420. The control valve body 410 is provided with a sliding slot 430 along its axial direction. The sliding slot 430 matches with the control slider 420 to enable the control slider 420 to slide along the sliding slot 430. Further, one end of the control valve body 410 defines a catheter insertion opening 440, through which proximal end of the inner tube 100 inserts into the control valve 400 and fixedly coupled to the control valve body 410 while the proximal end of the outer tube 200 is coupled to the control slider 420, for example, by adhesive bonding or snap-fitting. With this arrangement, movements (e.g., backward retraction or forward push) of the outer tube 200 relative to the inner tube 100 is able to be controlled by controlling sliding of the control slider 420. In this way, the control valve 400 is able to control expansion or contraction of the flow blocking member 300, thus simplifying operations involved in the surgical procedure, shortening the operation time and providing convenience for repeat operations. Optionally, proximal end of the outer tube 200 includes a stress dispersion tube 220. The stress dispersion tube 220 flares towards the proximal end. That is, the distal end of the stress dispersion tube 220 has a diameter equal to the diameter of the outer tube 200, and the proximal end of the stress dispersion tube 220 has a diameter greater than the diameter of the outer tube 200. With this arrangement, portion of the outer tube 200 configured to couple the control slider 420 has a widened diameter, and the flaring stress dispersion tube 220 helps in dispersing a drive force exerted by the control slider 420 on the outer tube 200, enabling to achieve a more reliable control of outer tube 200 by the control slider 420. In alternative embodiments, it is also possible to couple the outer tube to the control valve body 410, with the inner tube 100 being coupled to the control slider 420. Other direct or indirect coupling designs are also possible, and the present application is not limited in this regard.

Referring to FIG. 6, in one preferred embodiment, a groove 110 is disposed along the outer circumference of the inner tube 100. The shape of the groove 110 matches with the shape of the flow blocking element 300 in the collapsed configuration so as to accommodate the flow blocking element 300. The matching in shape includes that the groove 110 is dimensioned equal to or greater than the flow blocking member 300 in the collapsed configuration. Optionally, the groove 110 is an annular groove surrounding the inner tube 100. When fully collapsed, the flow blocking member 300 can be embedded in the groove 110. Preferably, the groove 110 have a length that is equal to or greater than a length of the flow blocking member 300 in the collapsed configuration so that the flow blocking member 300 can be entirely accommodated in the groove 110. The groove 110 allows an even smaller gap between the outer tube 200 and the inner tube 100, thus additionally reducing the proportion of the cross-sectional area of the tube accounting for the cross-sectional area of the entire flow blocking catheter, and increasing the proportion of the cross-sectional area of inner lumen (i.e., the inner lumen of inner tube 100) accounting for the cross-sectional area of the entire flow blocking catheter. The groove 110 allows the flow blocking catheter to maintain a constant outer diameter throughout its whole length. This can avoid damage to the flow blocking member 300 when the flow blocking catheter is passing through a tortuous blood vessel. Moreover, the locations of the inner tube 100 where the flow blocking member 300 is attached to the inner tube 100 are spaced from the central axis of the inner tube 100 by substantially equal radial distances, which is helpful in maintaining concentricity, and avoiding eccentricity, of the flow blocking member 300 during its expansion. As a result, the uniformity of the attachment of flow blocking member 300 to the blood vessel wall is able to be achieved, so as to reduce the risk of blood leakage.

Referring to FIG. 3, in one preferred embodiment, one end of the flow blocking member 300 is attached to the outer circumference of the inner tube 100, for example, by adhesive bonding or welding, and the other end of the flow blocking member 300 is a free end. Optionally, the first end 310 (e.g., the proximal end) of the flow blocking member 300 is attached to an outer surface of the inner tube 100, with its second end 320 (e.g., the distal end) being a free end. In this case, in an expanded configuration of the flow blocking member 300, the second end 320 is spaced from the distal end of the inner tube 100 by a distance in the range of 0-500 mm. It is to be understood that the second end 320 should not extend out of the distal end of the inner tube 100. That is, the flow blocking member 300 is entirely located closer to the operator than the distal end of the inner tube 100.

As shown in FIG. 7, preferably, the flow blocking catheter further includes a securing film 500 attached to the exterior of the proximal end of the flow blocking member 300 so as to cover at least part of the flow blocking member 300 and part of the inner tube 100. The attachment of the securing film 500 to the flow blocking member 300 and the inner tube 100 may be accomplished by, for example, adhesive bonding or heat shrinkage. The securing film 500 allows to strenghen the fixation between the flow blocking member 300 and the inner tube 100. Optionally, the securing film 500 has an axial length ranging from 1 mm to 10 mm. It is to be noted that, in some embodiments, the flow blocking member 300 may be first attached to the inner tube 100 (e.g., by adhesive bonding or welding), and the securing film 500 may be then applied as a secondary reinforcing fixture for enhancing the reliability of the fixation. In other embodiments, the flow blocking member 300 may be dimensioned to have a dimensional fit or an interference fit with the inner tube 100 and is secured to the inner tube 100 by the securing film 500. However, the present application is not limited in this regard.

Referring to FIG. 8, in another preferred embodiment, the flow blocking member 300 is attached to the outer circumference of the inner tube 100 at two ends by, for example, adhesive bonding, welding or a securing film. Optionally, the two ends of the flow blocking member 300 are spaced apart by a certain distance. With this arrangement, when expanded, the flow blocking member 300 appears like a fusiform shape, which allows to achieve a better contact with the blood vessel wall and thus an improved blood flow blocking effect. In addition, the shape of the flow blocking element 300 is more stable under the impact of blood flow, additionally reducing the risk of blood leakage. Of course, in some other embodiments, it is also possible to arrange the two ends of the flow blocking member 300 adjacent to, or even overlapping with each other. In these cases, the flow blocking member 300 in the expanded configuration assumes a shape resembling the Greek letter Ω, while still providing similar benefits. Therefore, the present application is not limited the distance between two ends of the flow blocking member 300.

In summary, the flow blocking catheter of the present application comprises an inner tube, a flow blocking member and an outer tube, the flow blocking member being self-expandable and sleeved on an exterior of the inner tube, at least a proximal end of the self-expandable flow blocking member attached to an outer circumference of the inner tube, the outer tube movably sleeved on the exterior of the inner tube to restrict an expansion of the flow blocking member. With this configuration, expansion of the flow blocking member is able to be controlled simply by pushing/retracting the outer or inner tube, which allows to achieve a fast shifting between different configurations, relocatability during a surgical procedure, and simple and time-saving operation. In addition, the flow blocking member is able to occlude blood flow with a controllably expansion, thereby lowering stimulation to the wall of the blood vessel while avoiding the problem of easy bursting arising from the use of a balloon. Moreover, the flow blocking member has a small thickness when in a collapsed configuration, allowing an increased inner diameter of the catheter at a given outer diameter of the flow blocking catheter and thus making it applicable to the treatment of large blood clots or passsage of instruments.

The description presented above is merely a few preferred embodiments of the present application and does not limit the protection scope of present application in any sense. Any change and modification made by those of ordinary skill in the art based on the above teachings fall within the protection scope of the appended claims

What is claimed is:

1. A flow blocking catheter, comprising:
an inner tube;
a self-expandable flow blocking member sleeved on an exterior of the inner tube, at least a proximal end of the self-expandable flow blocking member attached to an outer circumference of the inner tube; and
an outer tube movably sleeved on the exterior of the inner tube to restrict an expansion of the flow blocking member.

2. The flow blocking catheter of claim 1, wherein the flow blocking member comprises a support frame, wherein at least an proximal end of the support frame is attached to the outer circumference of the inner tube and the support frame is self-expandable.

3. The flow blocking catheter of claim 2, wherein the flow blocking member further comprises a flow blocking membrane attached to the support frame.

4. The flow blocking catheter of claim 1, wherein the flow blocking member has its one end attached to the outer circumference of the inner tube and the other end to be a free end, or the flow blocking member has opposing ends attached to the outer circumference of the inner tube.

5. The flow blocking catheter of claim 1, wherein the outer circumference of the inner tube is provided with a groove that matches with a shape of the flow blocking member in a collapsed configuration to accommodate the flow blocking member.

6. The flow blocking catheter of claim 1, further comprising a control valve configured to drive the outer or inner tube to move relative to the inner or outer tube.

7. The flow blocking catheter of claim 6, wherein the control valve comprises a control valve body and a control slider coupled to the control valve body, the control slider configured to be axially slidable, and wherein: the control valve body is coupled to a proximal end of the inner tube with the control slider being coupled to a proximal end of the outer tube; or the control valve body is coupled to the proximal end of the outer tube with the control slider being coupled to a proximal end of the inner tube.

8. The flow blocking catheter of claim 1, wherein both or either of the inner tube and the outer tube is a single-layered tube made of macromolecular material.

9. The flow blocking catheter of claim 1, wherein both or either of the inner tube and the outer tube has a structure comprising at least two layers, in which both or either of a first layer and a second layer from inside to outside is made of macromolecular material.

10. The flow blocking catheter of claim 1, wherein both or either of the inner tube and the outer tube has a structure comprising at least two layers, in which a second layer from inside to outside comprises one or more selected from the group consisting of braided structure, coil, and cut hypotube.

11. The flow blocking catheter of claim 1, wherein each of the inner and outer tubes has a triple-layered structure.

12. The flow blocking catheter of claim 1, wherein the inner tube comprises a first radiopaque ring disposed at a distal end of the inner tube.

13. The flow blocking catheter of claim 12, wherein the inner tube further comprises a second radiopaque ring disposed at a location of the inner tube where the flow blocking member is attached to the inner tube.

14. The flow blocking catheter of claim 12, wherein the flow blocking member has one end attached to the outer circumference of the inner tube and the other end to be an free end, and wherein the flow blocking member further comprises a third radiopaque ring disposed at the free end thereof.

15. The flow blocking catheter of claim 1, wherein the flow blocking member comprises at least one selected from the group consisting of mesh structure, open-loop structure and spiral structure, and wherein the flow blocking member is fabricated by braiding, winding or cutting.

16. The flow blocking catheter of claim 15, wherein the mesh structure is braided from 1 to 64 filaments, wherein the filament is at least one selected from the group consisting of regular filament, radiopaque filament and composite filament, the regular filament made of at least one selected from the group consisting of nickel-titanium alloy, cobalt-chromium alloy, stainless steel and macromolecular material, the radiopaque filament made of at least one selected from the group consisting of radiopaque metal, alloy of radiopaque metals and macromolecular material containing a radiopaque agent, the composite filament formed by a radiopaque core filament combined with a regular filament.

17. The flow blocking catheter of claim 1, further comprising a securing film that is secured and attached to an exterior of the proximal end of the flow blocking member and at least covers a part of the flow blocking member and a part of the inner tube.
